(19) United States
(12) Patent Application Publication
(10) Pub. No.: US 2024/0225846 A1
(43) Pub. Date: Jul. 11, 2024

(54) DIGITAL BALL POINT TOTAL HIP ARTHROPLASTY SMART IMPLANT

(71) Applicant: Orthosoft ULC, Montreal (CA)

(72) Inventor: Varun Jayant Oak, Tampa, FL (US)

(21) Appl. No.: 18/404,662

(22) Filed: Jan. 4, 2024

Related U.S. Application Data
(60) Provisional application No. 63/437,310, filed on Jan. 5, 2023.

(51) Int. Cl. A61F 2/34; A61B 5/00; A61B 5/11

(52) U.S. Cl. CPC A61F 2/34; A61B 5/1121; A61B 5/4851; A61B 5/686; A61F 2002/3611

(57) ABSTRACT

Various aspects of methods, systems, and use cases may be used to prevent dislocation of a joint replacement implant by limiting movement of the joint replacement implant to safe zones of movement. In some examples, the joint replacement implant includes a ball, an intermediate shell adapted to receive the ball and engage with a shell mobility structure protruding from the ball, and an outer shell adapted to receive the intermediate shell and provide fixation of the implant within a portion of a joint of a patient. For example, the interaction between the ball, the intermediate shell, and the outer shell prevent dislocation of the ball, and therefore dislocation of the joint replacement implant.

### CLAIM OF PRIORITY

This application claims the benefit of U.S. Provisional Patent Application Ser. No. 63/437,310, filed on Jan. 5, 2023, the benefit of priority of which is claimed hereby, and which is incorporated by reference herein in its entirety.

### BACKGROUND

The hip joint is a ball and socket joint. The socket is formed by part of the pelvis bone, while the ball is the femoral head. The surfaces of the bones are covered with articular cartilage to cushion the ends of the bones and provide ease of motion. When the hip joint is damaged, or is causing chronic hip pain, a total hip replacement (total hip arthroplasty) may be used. Hip joint damage or chronic pain may be caused by arthritis, disease, fractures, among others. Total hip arthroplasty may be used to replace portions of the hip joint with implants.

### OVERVIEW

The most common problem with total hip replacement is dislocation. Dislocation can occur due to improper cup placement. Improper cup placement usually results in the extremes of range of motion. Movements in the extremes of range of motion can lead to another common problem-impingement. Impingement occurs when the femoral head (ball of the hip) pinches up against the acetabulum (cup of the hip).

To avoid impingement, the present design determines the implant position using the range of motion of the native joint. The hip implant includes a full ball with a monoblock stem that is attached to the ball. The hip implant includes an intermediate shell (e.g., a metal shell) that covers most of the ball. For example, about one fourth of the surface of the ball remains exposed by the intermediate shell.

To solve problems with dislocation, the hip implant includes a safety mechanism created due to the shape of the intermediate shell. For example, the safety mechanism is created due to the shape of the shell with chevrons that connect with the chevrons, or oval ring structure, on the intermediate shell to move it to cover the ball from movement in the extreme ranges of movement.

The design of the hip implant shell improves the arc of motion covering the entire range of motion and avoids impingement and dislocation from occurring. The design and method used herein can remove the need for objectively measuring version and/or inclination (or even using robotics or navigation or templating). However, anteversion and inclination are parameters that are needed to ensure a full range of motion without dislocation.

Therefore, the safety zone can be marked using a digital sensor design. The digital sensor design will map the zone of movement of the hip joint with the center of the native hip as 0,0,0 in the cartesian coordinate system. This will create a three-dimension (3D) map that will cover how much the native hip moves in extension, ab-duction, ad-duction, internal rotation, and external rotation. The 3D map will be translated into a two-dimension (2D) model that will give an oval shaped safe zone.

The hip implant can include a digital gyroscope at its center. The movement of the implant can be matched to cover more area than the native hip to stay in the safe zone. The hip implant solves the issue of dislocation as stated above and it can be implied that if the implant has a wider safe zone than the native hip then it will be in the correct anteversion and inclination planes.

Trunnionosis is the wear of the femoral head-neck interface and can lead to failure of total hip replacements. The present design includes an implant neck and head in a monoblock design so there won't be surface related wear and tear at the neck-head interface.

The current design can also reduce implant wear and tear because the ball is in contact with the surfaces on the side and not in the traditional top portion of the head of the hip implant. The implant can also include one or more liners. For example, the hip implant can include a liner between the ball and the intermediate shell and/or the between the intermediate shall and the outer shell. Such a configuration can reduce backside wear of the implant.

Such solutions can provide a variety of additional advantages. The hip implant allows a patient the freedom to move in any direction like a native hip joint without worrying that the hip might dislocate. The digital mapping of the safe zone allows surgeons to perform the total hip replacement without worrying about the cup placement accuracy (including anteversion and inclination). The pelvic tilt and sacral slope will not affect dislocation as the ball is held along most of its surface within the liner and intermediate shell. The existing total hip replacement mechanisms are a hemispherical cup in which only the ball rotates resulting in covering less than half of the ball's surface in contact with the cup. With the present hip implant the traditional ball and socket mechanism is replaced with a full shell and liner in a liner that has support proximally and distally. The femoral shaft to neck angulation can also be adjusted to ensure the femoral anteversion is maintained. There is also no need for defining the acetabular anteversion or inclination because the native hip center will match with the implant hip center and map the safety zone of movement. The battery operated gyroscope and accelerometers in the femoral head will continue giving feedback on the safe zone and therefore implant loosening or position changes with time. The gyroscope and accelerometers can also give feedback on walking speeds, angulations, and other parameters.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A illustrates a system for assessing a range of motion to determine a safe zone for a hip implant, prior to implantation, in accordance with at least one example of this disclosure.

FIG. 1B illustrates a system for assessing a range of motion to determine a safe zone for a hip implant, after implantation, in accordance with at least one example of this disclosure.

FIG. 2A illustrates the range of motion safe zone of a native hip in accordance with at least one example of this disclosure.

FIG. 2B illustrates the range of motion safe zone of a hip implant in accordance with at least one example of this disclosure.

FIG. 2C illustrates various zones of a hip implant in comparison to a native implant in accordance with at least one example of this disclosure.

FIG. 3A illustrates a hip implant system in accordance with at least one example of this disclosure.

FIG. 3B illustrates a hip implant system inserted into the hip in accordance with at least one example of this disclosure.

FIG. 4A illustrates an example graph of patient's hip movement in accordance with at least one example of this disclosure.

FIG. 4B illustrates a first cross section view of an intermediate shell of a hip implant system in accordance with at least one example of this disclosure.

FIG. 4C illustrates a second cross section view of a hip implant system with an intermediate shell in accordance with at least one example of this disclosure.

FIG. 4D illustrates a cross section view of a hip implant system in a neutral position in accordance with at least one example of this disclosure.

FIG. 4E illustrates a cross section view of a hip implant system in an engaged position in accordance with at least one example of this disclosure.

FIG. 4F illustrates a cross section view of a hip implant system in a rotated position in accordance with at least one example of this disclosure.

FIG. 5A illustrates a hip implant system in accordance with at least one example of this disclosure.

FIG. 5B illustrates another view of the hip implant system of FIG. 5A in accordance with at least one example of this disclosure.

### DETAILED DESCRIPTION

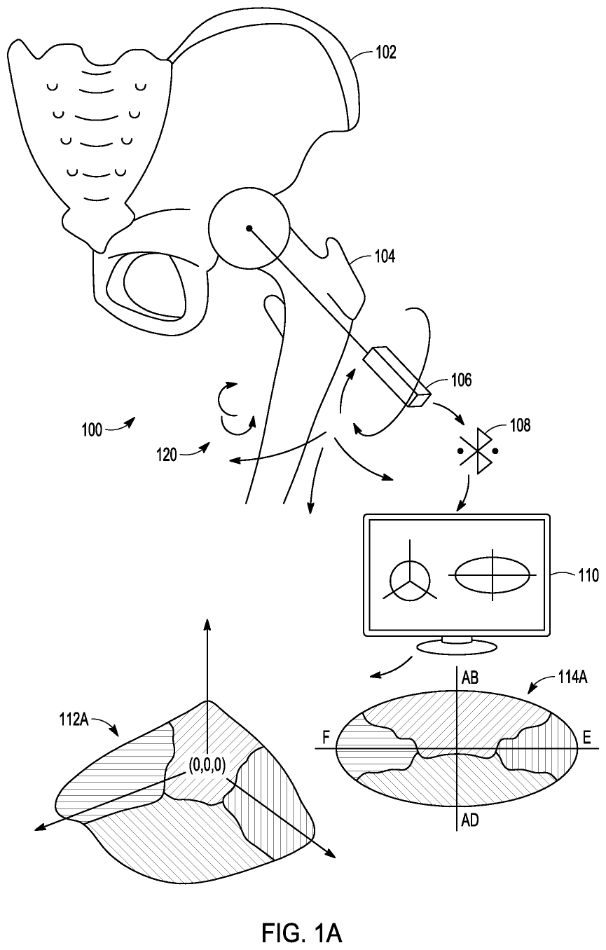

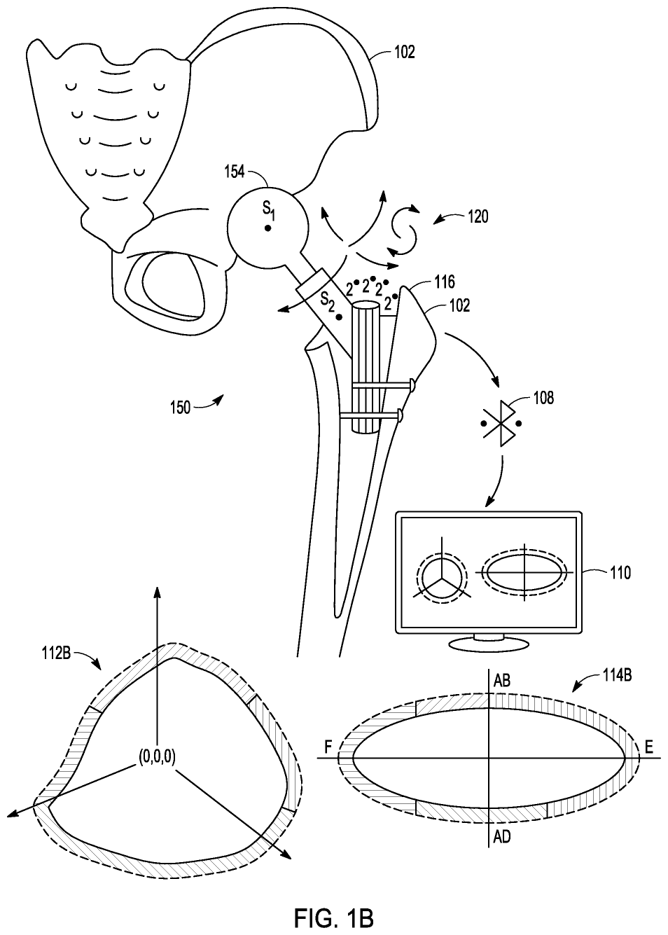

FIG. 1A illustrates an example system 100 for assessing a range of motion to determine a safe zone for a hip implant, prior to implantation. The system includes a sensor array probe 106 and a computer 110. The sensor array probe 106 is drilled into the femoral head of a femur 104. For example, the sensor array probe 106 can be implanted into the femoral head of the patient to assist in assessing range of motion in a pre-operative planning procedure. In another example, the sensor array probe 106 can be embedded into a femoral implant, such as a trial implant used in planning a total hip arthroplasty.

For example, to begin, the patient lays on a table in a lateral decubitus position and is cleaned and draped. The hip and leg should be allowed to move freely in all directions without constraints. The patient is anaesthetized with general anaesthesia or a spinal anaesthesia. A lateral or posterolateral approach can be used during the procedure. After creating a skin incision, keeping the muscle and capsule intact, the sensor array probe 106 is used.

In an example, the sensor probe 106 includes a radiolucent tip that is inserted into the center of the femoral head of the femur 104 under fluoroscopic guidance. In an example, a second sensor is positioned lateral to the first sensor to create two points in a line. In the illustrated example, the sensor probes transmit movements through Bluetooth transmission 108. After the sensor 106 is turned on, the surgeon and/or assistant move the limb through its maximum range of motion 120. The full range of motion includes full flexion, full extension, full ab-duction, full ad-duction, full internal rotation, and full external rotation.

A center of the femoral head, or a point near the center of the head, is considered the center (0,0,0) in a three-dimensional cartesian coordinate system. The second sensor moves in relation to the sensor array probe 106. As such, the sensor array creates a three-dimensional image of the entire range of motion in a computer 110. The range of motion image can be maintained in a three-dimension image 112a or converted to a two-dimensional image 114a.

The three-dimensional image 112a and the two-dimensional image 114a are a graphical or pictorial representation of the range of motion of the patient's hip joint. The three-dimensional image 112a and the two-dimensional image 114a can be registered and stored in the computer 110. The range of motion of the native hip is considered the safe zone.

The patient's joint has certain anteversion (femoral and acetabular) and inclination, and therefore the native hip moves in a specific range of motion. By determining the range of motion, the hip implant can match or outperform the range of motion. Matching or outperforming the range of motion will allow the correction selection for the anteversion and inclination when setting the implant, and will increase the changes of a successful total hip replacement.

In an example, after the range of motion has been determined, the skin is opened to conduct capsulotomy and large osteophyte removal. Once this procedure is completed, the range of motion exercises are repeated to see how the digital safe zone is created without the abductor tension and capsular tension.

FIG. 1B illustrates an example system 150 for assessing a range of motion to determine a safe zone for a hip implant 154, after implantation.

In an example, the hip implant 154, or a trial implant, is switched on and positioned in the acetabulum and the femoral stem 116. Switching on the hip implant 154 includes turning on a sensor embedded in the hip implant 154. In an example, the sensors of the hip implant 154 can include accelerometers, for example, two accelerometers. In an example, the sensors of the hip implant 154 include a gyroscope. In an example, the sensors of the hip implant 154 include one or more accelerometers and a gyroscope. The accelerometer(s) and gyroscope may be located in the head and lateral to the head.

After the hip implant 154 is positioned, the surgeon and/or the assistant move the hip implant 154 through the entire range of motion 120. The entire range of motion includes full flexion, full extension, full ab-duction, full ad-duction, full internal rotation, and full external rotation. In an example, the sensors transmit data to a computer 110. The data can be transmitted via Bluetooth 108. Using the sensor data, the computer 110 creates a three-dimensional image of the entire range of motion in a computer 110. The range of motion image can be maintained in a three-dimension image 112b or converted to a two-dimensional image 114b. If the implant safe zone of the is wider than the native safe zone, the implant positioning is accurate. In such an example, when the implant positioning is accurate, the anteversion or inclination does not need to be measured. If the implant is not positioned to generate the correct safe zone, the hip implant 154, or a trial hip implant, can be adjusted by the surgeon in order to match the safe zone or widen it.

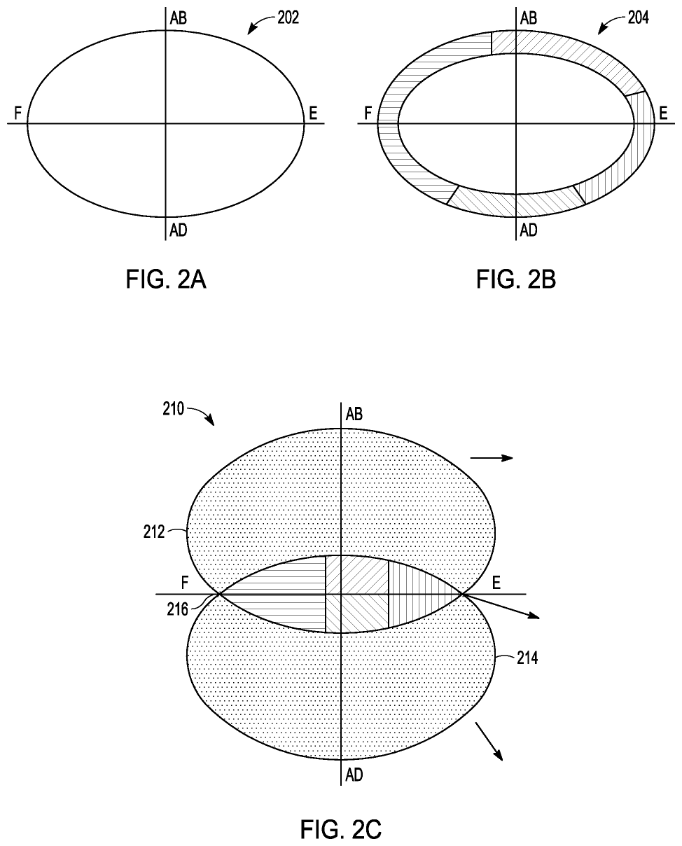

FIGS. 2A-2C illustrate example ranges of motion. The range of motions can be considered in 2D where the 3D model is converted in to a 2D model by the computer or the 3D model can itself be used.

FIG. 2A illustrates an example range of motion safe zone 202 of a native hip. The range of motion safe zone 202 includes full flexion (F), full extension (E), full ab-duction (AB), and full ad-duction (AD).

FIG. 2B illustrates an example range of motion safe zone 204 of a hip implant. The range of motion safe zone 204 of the hip implant includes full flexion (F), full extension (E), full ab-duction (AB), and full ad-duction (AD). Ideally, the range of motion safe zone 204 of the hip implant extends slightly beyond the range of motion safe zone 202 of a native hip. For example, the range of motion safe zone 204 is 10 degrees to 20 degrees greater than the range of motion safe zone 202 of the native hip.

FIG. 2C illustrates example zones 210 of a hip implant in comparison to a native implant. There are three zones identified in the comparison between the hip implant and the native implant. The three zones include a danger zone 212, a wasted zone 214, and a safe zone 216.

The danger zone 212 is the zone where the native hip wants to move but the hip implant restricts movement. The wasted zone 214 is the area where the hip implant wants to move, but the native hip anatomy restricts movement. Movement in the wasted zone 214 can cause impingement, which can lead to implant loosening, damage, and poor range of motion outcomes. The impingement occurs because the implant version and inclination do not match the expected version and inclination of the native hip.

The safe zone 216 is the ideal zone in which the hip implants range of motion matches the native hip range of motion. This is where the motion of the implant occurs. If the implant matches the native hip alignment in terms of version, inclination, and height, it can be implied that a safe zone is created. In an example, an ideal safe zone 216 is when an implant range of motion (implant safe zone) is concentrically wider than the native hip plant range of motion (native safe zone).

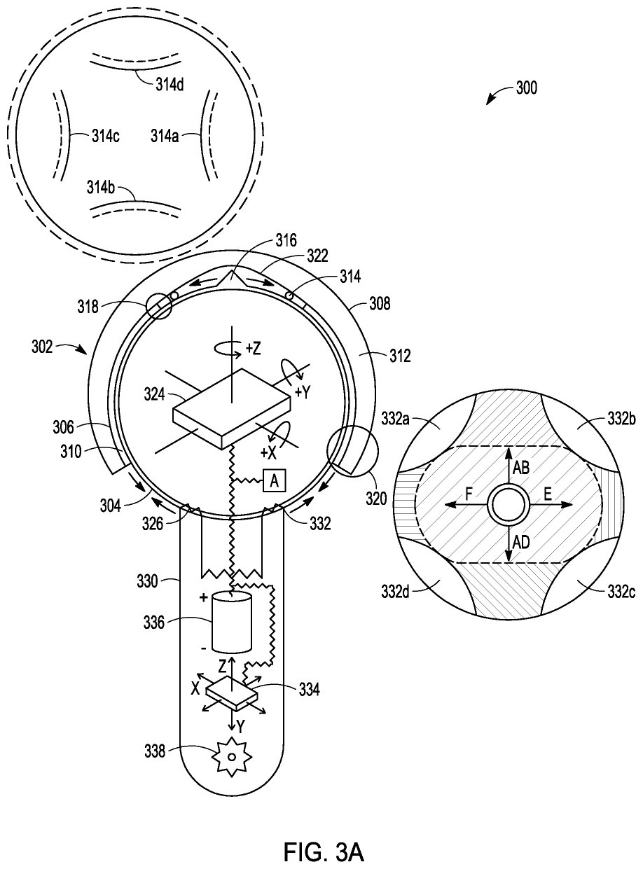

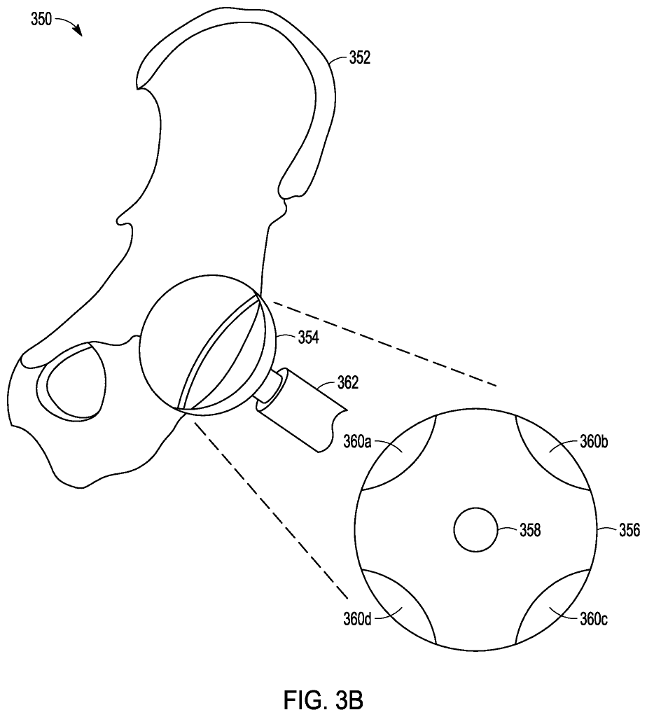

FIG. 3A illustrates an example hip implant system 300. The hip implant system 300 includes a head 302 and a neck 330. In the illustrated example, the head 302 includes a ball 304, an intermediate shell 306, and an outer shell 308. The outer shell 308 operates as an acetabular cup that is implanted in an acetabulum of a pelvis bone of the patient. The ball 304 and the intermediate shell 306 are sized and shaped to interface with the outer shell 308.

In the illustrated example, the ball 304 is substantially round. In an example, the ball 304 is metal. In an example, the ball 304 includes a monoblock neck 326. The monoblock neck 326 extends from the ball 304 to attach the ball 304 to the neck 330. In the illustrated example, the ball 304 includes a shell mobility structure 316. The shell mobility structure 316 of the ball 304 is positioned on the proximal side of the ball 304, opposite the monoblock neck 326. For example, the shell mobility structure 316 and the monoblock neck 326 share the same axis of the ball 304 (e.g., a longitudinal axis running the length of the neck 330 through the center of the ball 304 and out through the shell mobility structure 316). In an example, the ball 304 includes a sensor 324. The sensor 324 may be a gyroscope and/or an accelerometer.

The intermediate shell 306 is positioned between the ball 304 and the outer shell 308. In an example, the intermediate shell 306 is metal. In an example, the intermediate shell 306 extends over a portion of the surface area of the ball 304. For example, the intermediate shell 306 does not cover the entire ball 306 but extends over at least half of the surface area of the ball 306. In the illustrated example, the intermediate shell 306 includes a conical shape on an exterior surface of a proximal side of the intermediate shell 306.

In the illustrated example, the intermediate shell 306 includes chevrons 314 extending inward from an interior surface of the intermediate shell 306. For example, the chevrons 314 includes four chevrons 314a, 314b, 314c, and 314d. In an example, the chevrons 314 are evenly spaced along an interior surface of the intermediate shell 306. The shell mobility structure 316 interacts with the chevrons 314 to shift the rotational position of the intermediate shell 306 within the outer shell 308. For example, as the ball 306 moves around, the rotation of the intermediate shell 306 via engagement of the chevrons 314 from extreme movement prevents dislocation of the ball 304. In an example, the spacing between the chevrons 314 compared to the size of the shell mobility structure 316 prevents the shell mobility structure 316 from going between the chevrons 314. For example, the chevrons 314 cause movement of the ball 304, and therefore the neck 330 (and thus the native femur) that is beyond the safe zone of the native femur to be maintained within the safe zones (e.g., from FIGS. 1A-1B and FIGS. 2A-2C) of the hip implant system 300 due to the rotation of the intermediate shell 306. For example, the chevrons 314 can allow a range of motion about 10 degrees to 20 degrees greater than the native joint safe zone range of motion to provide a safety mechanism, preventing dislocation of the hip implant system 300. For example, the maximum normal hip joint movements for the native joint are as follows:

| Motion | Max range of the native hip |
|---|---|
| Abduction | 100 degrees |
| Adduction | 45 degrees |
| Internal Rotation | 50 degrees |
| External Rotation | 80 degrees |
| Extension | 25 degrees |
| Flexion | 150 degrees |

Accordingly, the range of motion of the hip implant is 10 degrees to 20 degrees greater than the maximum ranges in the table.

In an example, an inner liner 310 is positioned between the ball 304 and the intermediate shell 306. For example, the inner liner 310 is a spherical frustum. In an example, the inner liner 310 fills the entire space between the ball 304 and the intermediate shell 306. For example, the inner liner 310 has an inner dimeter corresponding to an outer diameter of the ball 304 and an outer diameter corresponding to an inner diameter of the intermediate shell 306. In an example, the inner liner 310 includes a plurality of liner sections connected to the ball 304, the intermediate shell 306, and/or another of the plurality of liner sections. For example, plurality of liner sections may be positioned corresponding to the chevrons 314. In an example, there are four liner sections. In the illustrated example, the inner liner 310 has an end 318 that does not extend all the way to the chevrons 314. For example, the inner liner 310 extends around the ball 304, leaving a space between the liner end 318 and the chevrons 314, such that the inner liner 310 and the chevrons 314 do not touch. In an example, the inner liner 310 moves if the shell mobility structure 316 engages with chevrons 314. In an example, the inner liner 310 is connected to the intermediate shell 306, such that when the shell mobility structure 316 engages with chevrons 314, the inner liner 310 and intermediate shell 306 move together.

The outer shell 308 surrounds the intermediate shell 306. In an example, the outer shell 308 is metal. In an example, the outer shell 308 extends over a portion of the surface area of the ball 304. For example, the outer shell 308 does not cover the entire ball 304 but extends over at least half of the surface area of the ball 304. In the illustrated example, the outer shell 308 extends around the entirety of the intermediate shell 306. In an example, an outer liner 312 is positioned between the intermediate shell 306 and the outer shell 308. For example, the outer liner 312 is a spherical frustum. In an example, the outer liner 312 fills the entire space between the intermediate shell 306 and the outer shell 308. In an example, the outer liner 312 has an inner dimeter corresponding to an outer diameter of the intermediate shell 306 and an outer diameter corresponding to an inner diameter of the outer shell 308. In an example, the outer liner 312 includes a plurality of liner sections connected to the intermediate shell 306, the outer shell 308, and/or another of the plurality of liner sections.

In the illustrated example, the inner liner 310, the intermediate shell 306, the outer liner 312, and the outer shell 308 all have the same edge 320, when the implant is in a neutral position. For example, the inner liner 310, the intermediate shell 306, the outer liner 312, and the outer shell 308 each have an edge that is flush with the other edges when the implant is in a neutral position (as shown). Accordingly, the inner liner 310, the intermediate shell 306, the outer liner 312, and the outer shell 308 all extend around the ball 304 as a semi-circle, about 180 degrees. In an example, the inner liner 310, the intermediate shell 306, the outer liner 312, and the outer shell 308 extend around the ball 304 more than a semi-circle, such as about 190 to 220 degrees. In an example, the inner liner 310, the intermediate shell 306, the outer liner 312, and the outer shell 308 extend around the ball 304 about 190 to 200 degrees. In an example, the outer shell 308 and/or the outer liner 312 extend further around the intermediate shell 306 than the intermediate shell 306 extends around the ball 304. The outer shell 308 and/or the outer liner 312 extending further around the intermediate shell 306 may allow the outer shell 308 and/or the outer liner 312 to remained fixed, while rotation of the intermediate shell 306 does not cause the intermediate shell 306 to extend outside of the outer shell 308 and/or the outer liner 312. Movement of the intermediate shell 306 within the outer shell 308 is caused by interaction between the chevrons 314 along portions of the inner surface of the intermediate shell 306 and the shell mobility structure 316. The movement of the intermediate shell 306 within the outer shell 308 operates to prevent dislocation of the ball 304 at the extremes of the range of motion.

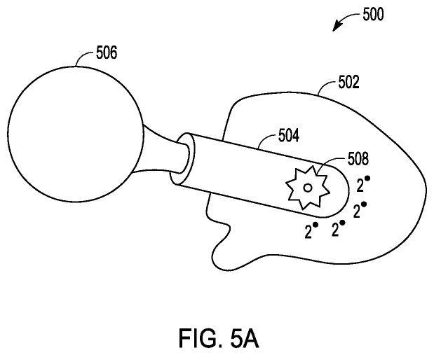

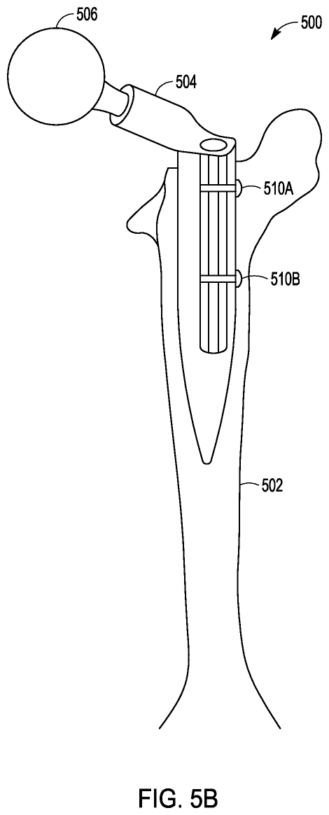

The neck 330 design to be attached via a ratchet mechanism (described below) to a femoral stem, and the femoral stem is inserted into the native femur after removal of the native femoral head (as shown in FIGS. 5A-5B). In the illustrated example, the neck 330 include a monoblock neck interface 332. For example, the monoblock neck interface 332 secures the monoblock neck 326 to the neck 330. In an example, the monoblock neck 326 secures to the monoblock neck interface 332 via a snapfit-lock. In the illustrated example, the monoblock neck interface 332 includes interfaces 332a, 332b, 332c, and 332d. The interfaces 332a, 332b, 332c, and 332d secure the monoblock neck 326 to the neck 330.

In an example, the neck 330 includes a sensor 326. The sensor may be a gyroscope and/or an accelerometer. In an example, the neck 330 includes a battery 336. The battery 336 may be a long lasting battery. In an example, the battery 336 and/or the sensor 326 includes a transmitter device. For example, the transmitter device transmits via Bluetooth.

In the illustrated example, the neck 330 includes a ratchet mechanism 338. For example, the ratchet mechanism 338 adjusts the neck 330 with respect to the femoral stem that extends into the femoral canal. For example, the ratchet mechanism 338 includes a gearing mechanism. For example, the femoral anteversion is adjusted using the ratchet mechanism 338. In an example, the ratchet mechanism 338 is adjustable in increments of about 1-2 degrees. In an example, the ratchet mechanism 338 is adjustable up to a maximum of about 20 degrees.

FIG. 3B illustrates an example of a hip implant 350 inserted into a pelvic bone 352. In an example, the hip implant 350 is a trial implant. The hip implant 350 includes a head 354 and a stem 362.

After a femoral canal is prepared, the hip implant 350 can be inserted into the femoral canal. For example, the stem 362 of the hip implant 350 is inserted into the femoral canal. Then, the surgeon positions the hip implant 350 into the acetabulum of the pelvic bone 352 of the patient. For example, the head 354 of the hip implant 350 is positioned in the acetabulum. In an example, additional preparations are done to adjust the depth of the hip implant 350, or the height of the acetabulum. The depth of the hip implant 350 is important to ensure good bone grasping and allow bone growth. In an example, reaming will only be done until bleeding cancellous bone is reach. In an example where the patient has abductor weakness, the surgeon may medialize more.

In an example, the surgeon selects a size of the head 354 based on the size of the acetabulum. For example, a diameter of the head 354 is selected that matches, or substantially matches, the diameter of the acetabulum. In an example, the head 354 includes an outer shell (as described above and below), and the diameter of the outer shell is used to match the diameter of the acetabulum. For example, the average adult acetabulum may have a diameter of about 6 cm, and a radius of about 3 cm. The ball may have a radius of about 1.5 cm to 2.5 cm. For example, the ball may have a radius of about 1.5 cm to 2 cm. A smaller ball would require more angular motion to achieve a similar distance traveled. In an example, a smaller ball would require more angular motion to achieve less distanced traveled, compared to a larger ball. The distance traveled, the sectoral distance, is determined using the following equation: θ/360*2πr=sectoral distance. Accordingly, to achieve the maximum degrees of motion based on the safe zone, the ball would have to travel different distances, as shown in the following table (in cm):

| | | | | |
|---|---|---|---|---|
| Ball Dimension | Radius | 1.5 | 2 | 2.5 |
| | Circumference | 9.4 | 12.6 | 15.7 |
| Sectoral distance | Flexion | 3.9 | 5.2 | 6.5 |
| covered | Extension | 0.7 | 0.9 | 1.1 |
| | Abduction | 2.6 | 3.5 | 4.4 |
| | Adduction | 1.2 | 1.6 | 2.0 |
| | Internal rotation | 1.3 | 1.7 | 2.2 |
| | External rotation | 2.1 | 2.8 | 3.5 |

In an example, the hip implant 350 is fixed using cemented fixation or cementless fixation. With cementless fixation, the exterior of the outer shell of the head 354 is made of a cementless trabecular material. In an example, when the hip implant 350 is an implanted using one or more screws to measure a range of motion of the implant compared to the safe zone range of motion of the native hip.

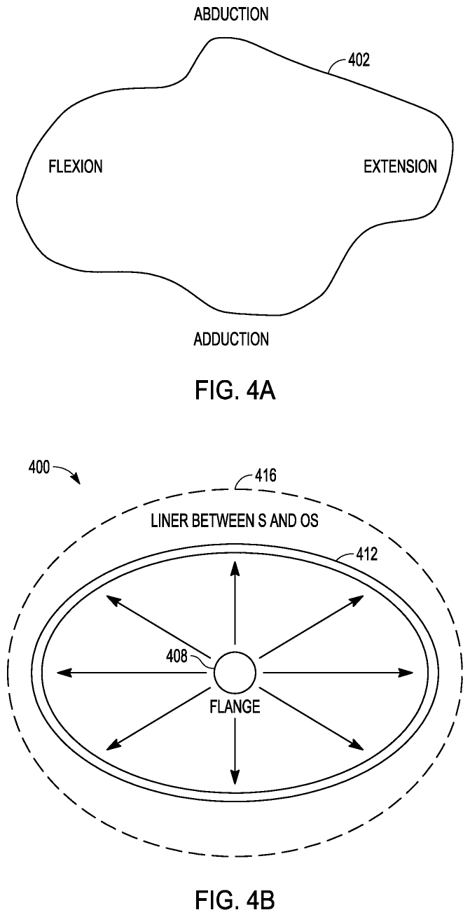

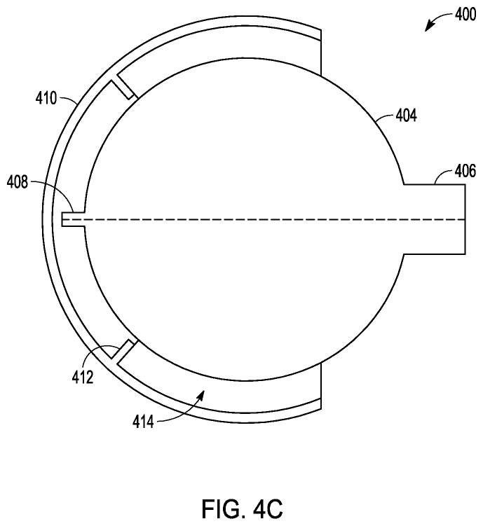

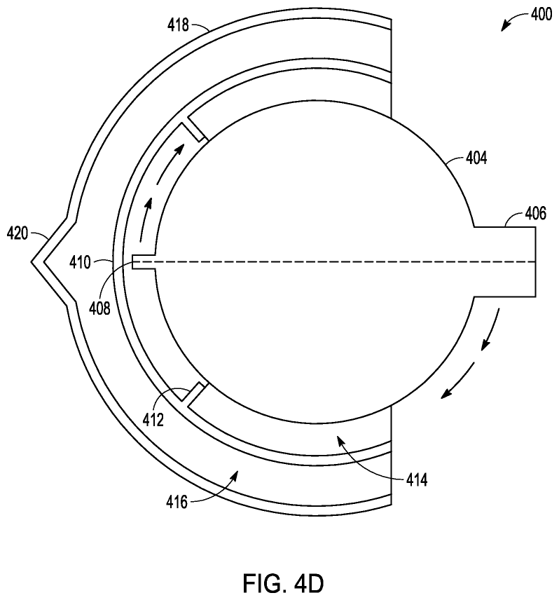

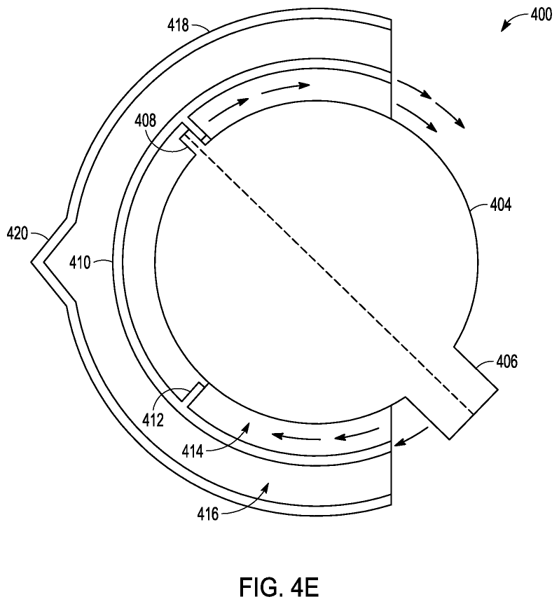

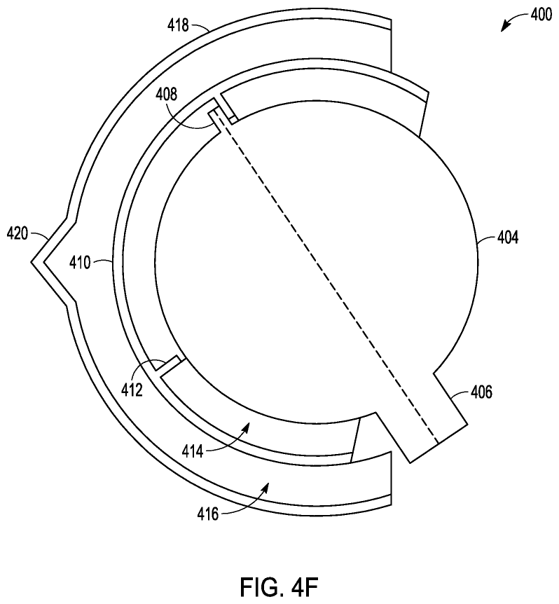

FIG. 4A illustrates an example graph 402 of patient's hip movement. For example, this illustrates the natural movement of the patient's hip joint. In an example, the graph 402 of the patient's hip movement is used to map an intermediate shell of a head of a hip implant. In another example, the graph 402 is compared to a graph obtained after implantation of a hip implant to ensure a safety zone of the implant is larger than the natural movement of the patient's hip joint.

FIGS. 4B-4F illustrate an example hip implant system 400. The hip implant system 400 includes a ball 404, an intermediate shell 410, and an outer shell 418. The outer shell 418 operates as an acetabular cup that is implanted in an acetabulum of a pelvis bone of the patient. The ball 404 and the intermediate shell 410 are sized and shaped to interface with the outer shell 418.

The ball 404 is substantially round. In the illustrated example, the ball 404 includes a monoblock neck 406 and a shell mobility structure 408. In an example, the ball 404 is made of metal. In the illustrated example, the monoblock neck 406 secures the ball 404 to a femoral stem of the hip implant system 400 (femoral stem is not illustrated for clarity). The shell mobility structure 408 extends from the ball 404 opposite the monoblock neck 406. For example, the shell mobility structure 408 extends from a central proximal surface of the ball 404. Accordingly, the monoblock neck 406 and the shell mobility structure 408 have the same axis of movement (e.g., a longitudinal axis running the length of the monoblock neck 406 through the center of the ball 404 and out through the shell mobility structure 408).

In an example, the surgeon selects a size of the hip implant system 400 based on the size of the acetabulum. For example, a diameter of the hip implant system 400 is selected that matches, or substantially matches, the diameter of the acetabulum. In an example, the diameter of the outer shell 418 is used to match the diameter of the acetabulum. The ball 404 has a diameter smaller than the outer shell 418. For example, the average adult acetabulum may have a diameter of about 6 cm, and a radius of about 3 cm. The ball 404 may have a radius of about 1.5 cm to 2.5 cm. For example, the ball 404 may have a radius of about 1.5 cm to 2 cm. A smaller ball would require more angular motion to achieve a similar distance traveled. In an example, a smaller ball would require more angular motion to achieve less distanced traveled, compared to a larger ball. The distance traveled, the sectoral distance, is determined using the following equation: θ/360*2πr=sectoral distance. Accordingly, to achieve the maximum degrees of motion based on the safe zone, the ball would have to travel different distances, as shown in the following table (in cm):

| | | | | |
|---|---|---|---|---|
| Ball Dimension | Radius | 1.5 | 2 | 2.5 |
| | Circumference | 9.4 | 12.6 | 15.7 |
| Sectoral distance | Flexion | 3.9 | 5.2 | 6.5 |
| covered | Extension | 0.7 | 0.9 | 1.1 |
| | Abduction | 2.6 | 3.5 | 4.4 |
| | Adduction | 1.2 | 1.6 | 2.0 |
| | Internal rotation | 1.3 | 1.7 | 2.2 |
| | External rotation | 2.1 | 2.8 | 3.5 |

The intermediate shell 410 is positioned between the ball 404 and the outer shell 418. In an example, the intermediate shell 410 receives the ball 404. In an example, the intermediate shell 410 is metal. In an example, the intermediate shell 410 extends over a portion of the surface area of the ball 404. For example, the intermediate shell 410 does not cover the entire ball 404 but extends over at least half of the surface area of the ball 404. In an example, the intermediate shell 410 is a partial sphere.

In the illustrated example, the intermediate shell 410 includes a shell mobility ridge 412. For example, the shell mobility ridge 412 extends inward from an interior surface of the intermediate shell 410. In the illustrated example, the shell mobility ridge 412 extends distally from an interior surface of the intermediate shell 410. In an example, the shell mobility ridge 412 is formed by edges of a void in the intermediate shell 412. In an example, the shell mobility ridge 412 is a ring type structure. For example, the shell mobility ridge 412 is an oval shape when projected on a 2D plane (e.g., as shown in FIG. 4B). The shell mobility structure 408 interacts with the shell mobility ridge 412. In an example, the shell mobility structure 408 has a height that is at least half the height of the shell mobility ridge 412, in order for the shell mobility structure 408 to engage with the shell mobility ridge 412. In an example, the shell mobility structure 408 interacts with the shell mobility ridge 412 to shift the rotational position of the intermediate shell 410. For example, as the ball 404 moves around, the rotation of the intermediate shell 410 via engagement of the shell mobility ridge 412 from extreme movement prevents dislocation of the ball 404. In an example, the shell mobility structure 408 engages with the shell mobility ridge 412 in any direction the shell mobility structure 408, and therefore the hip joint, moves. For example, the shell mobility ridge 412 causes movement of the ball 404, and therefore the monoblock neck 406 (and thus the native femur) that is beyond the safe zone of the native femur to be maintained within safe zones (e.g., from FIGS. 1A-1B and FIGS. 2A-2C) of the hip implant system 400 due to the rotation of the intermediate shell 410 thereby preventing dislocation. For example, the shell mobility ridge 412 can allow a range of motion about 10 degrees to 20 degrees greater than the native joint safe zone range of motion to provide a safety mechanism, preventing dislocation of the hip implant system 400. For example, the maximum normal hip joint movements for the native joint are as follows:

| Motion | Max range of the native hip |
|---|---|
| Abduction | 100 degrees |
| Adduction | 45 degrees |
| Internal Rotation | 50 degrees |
| External Rotation | 80 degrees |
| Extension | 25 degrees |
| Flexion | 150 degrees |

Accordingly, the range of motion of the hip implant is 10 degrees to 20 degrees greater than the maximum ranges in the table.

In an example, an inner liner 414 is positioned between the ball 404 and the intermediate shell 410. For example, the inner liner 414 is a spherical frustum. In an example, the inner liner 414 fills the entire space between the ball 404 and the intermediate shell 410. For example, the inner liner 414 has an inner dimeter corresponding to an outer diameter of the ball 404 and an outer diameter corresponding to an inner diameter of the intermediate shell 410. In an example, the inner liner 414 includes a plurality of liner sections connected to the ball 404, the intermediate shell 414, and/or another of the plurality of liner sections. For example, plurality of liner sections may be positioned corresponding to the shell mobility ridge 412. In an example, there are four liner sections. In the illustrated example, the inner liner 414 has an end that does not extend all the way to the shell mobility ridge 412. For example, the inner liner 414 extends around the ball 404, leaving a space between the liner end and the shell mobility ridge 412, such that the inner liner 414 and the shell mobility ridge 412 do not touch. In an example, the inner liner 414 moves if the shell mobility structure 408 engages with shell mobility ridge 412. In an example, the inner liner 414 is connected to the intermediate shell 410, such that when the shell mobility structure 408 engages with the shell mobility ridge 418, the inner liner 414 and intermediate shell 410 move together.

The outer shell 418 surrounds the intermediate shell 410. In an example, the outer shell 418 receives the intermediate shell 410. In an example, the outer shell 418 is metal. In an example, the outer shell 418 extends over a portion of the ball 404. For example, the outer shell 418 does not cover the entire ball 404 but extends over at least half of the surface area of the ball 404. In the illustrated example, the outer shell 418 extends around the entirety of the intermediate shell 410. In an example, the outer shell 418 is a partial sphere. In an example, an inner surface of the outer shell 418 is a partial sphere.

In an example, an outer liner 416 is positioned between the intermediate shell 410 and the outer shell 418. For example, the outer liner 416 is a spherical frustum. For example, the outer liner 416 fills the entire space between the intermediate shell 410 and the outer shell 418 and extends from the shell mobility ridge 412 to an edge of the intermediate shell 410 and the outer shell 418. In an example, the outer liner 416 has an inner dimeter corresponding to an outer diameter of the intermediate shell 410 and an outer diameter corresponding to an inner diameter of the outer shell 418. In an example, the outer liner 416 includes a plurality of liner sections connected to the intermediate shell 410, the outer shell 418, and/or another of the plurality of liner sections. For example, the outer liner 416 includes four liner sections. In the illustrated example, the intermediate shell 410, the outer liner 416, and the outer shell 418 all have the same edge when the hip implant system 400 is in a neutral position (as illustrated in FIG. 4D). For example, the intermediate shell 410, the outer liner 416, and the outer shell 418 each have an edge that is flush with the other edges when the hip implant system 400 is in a neutral position (as illustrated in FIG. 4D). For example, the intermediate shell 410, the outer liner 416, and/or the outer shell 418 extend around the ball 404 as a semi-circle, about 180 degrees. In an example, the intermediate shell 410, the outer liner 416, and/or the outer shell 418 extend around the ball 404 more than a semi-circle, such as about 190 to 220 degrees. In an example, the intermediate shell 410, the outer liner 416, and/or the outer shell 418 extend around the ball 404 about 190 to 200 degrees. In an example, the outer shell 418 and/or the outer liner 416 extend further around the intermediate shell 410 than the intermediate shell 410 extends around the ball 404. The outer shell 418 and/or the outer liner 416 extending further around the intermediate shell 410 may allow the outer shell 418 and/or the outer liner 416 to remained fixed, while rotation of the intermediate shell 410 does not cause the intermediate shell 410 to extend outside of the outer shell 418 and/or the outer liner 416.

In the illustrated example, the exterior of the outer shell 418 includes a fixation feature 420. The fixation feature 420 aids in fixing the outer shell 418 to an acetabulum of the patient. In the illustrated example, the fixation feature 420 is a conical shape located at a proximal apex of the outer shell 418. Similarly, the outer liner 416 includes a portion that engages with the fixation feature 420 of the outer shell 418. Accordingly, the outer liner 416 is fixed by the fixation feature 420, preventing movement of the outer liner 416. For example, the outer liner 416 may include a conical shaped portion to engage with the fixation feature 420 of the outer shell 418.

In an example, as the monobock neck 406 (e.g., by movement of a stem of the hip implant, such as by movement of the femur) causes rotation of the ball 404, the shell mobility structure 408 moves within the space defined by the shell mobility ridge 412 until the shell mobility ridge is engaged (as shown in FIG. 4E), if the ball 404 continues to move the shell mobility ridge 408 continues to engage the shell mobility ridge 412, causing rotation of the intermediate shell 410 and the inner liner 414 (as shown in FIG. 4F). In an example, if the ball 404 is rotated, such that the shell mobility structure 408 engages the shell mobility ridge 412, rotation of the ball 404 is not limited to the space defined by the shell mobility ridge 412. In an example, if the ball 404 is rotated, such that the shell mobility structure 408 engages the shell mobility ridge 412, the intermediate shell 410 is rotated upon engagement, while the outer liner 416 and the outer shell 418 remain stationary (as shown in FIG. 4F). However, rotation of the intermediate shell 410 may be limited. For example, rotation of the intermediate shell 410 may be limited such that the intermediate shell 410 does not extend beyond the outer shell 418. For example, rotation of the intermediate shell 410 may be limited by the monoblock neck 406 engaging with the intermediate shell 410 or the outer shell 418.

FIGS. 5A and 5B illustrate an example of a hip implant system 500.

The hip implant system 500 includes a native femur 502, an implant stem 504, and an implant head 506. The implant stem 504 and implant head 506 will be positioned based on the safe zones identified above. For example, the positioning of the implant is based on the safe zone of the native hip and/or the safe zone of the implant during testing.

In the illustrated example, the implant stem 504 includes a ratchet mechanism 508. For example, the ratchet mechanism 508 includes a gearing mechanism. In an example, the ratchet mechanism 508 is used to align the implant stem 504 with the native femur 502. For example, the femoral anteversion is adjusted using the ratchet mechanism 508. In an example, the ratchet mechanism 508 is adjustable in increments of about 1-2 degrees. In an example, the ratchet mechanism 508 is adjustable up to a maximum of about 20 degrees.

In an example, the implant stem 504 is locked into the native femur 502 with interlocking screws 510a and 510b. In an example, the implant head 506 is fixed to the acetabulum with a cemented or cementless manner. In an example, the implant stem 504 is fixed in the native femur 502 with a cemented or cementless manner. In an example, offset of the implant stem 504 is selected based on abductor tension, body weight of the patient, and how much medialization was done.

Once the implant stem 504 and implant head 506 are in a final position, the hip implant can be turned on. For example, sensors within the implant are turned on. The wound from the hip implant surgery is then closed in layers.

The above invention is discussed in reference to a total hip replacement, but the same concept can be applied to shoulder implant applications.

What is claimed is:

1. A joint replacement implant comprising:
a ball including a monoblock neck and a shell mobility structure protruding from a central proximal surface opposite the monoblock neck;
an intermediate shell adapted to receive the ball, the intermediate shell including a mobility ridge configured to engage with the shell mobility structure at various angular positions of the ball within the intermediate shell; and
an outer shell adapted to receive the intermediate shell, the outer shell including fixation features configured to secure the implant within a portion of a joint of a patient.

2. The joint replacement implant of claim 1, wherein the intermediate shell is a partial sphere.

3. The joint replacement implant of claim 2, wherein the mobility ridge extends distally from an inner surface of the intermediate shell.

4. The joint replacement implant of claim 3, wherein the mobility ridge forms an oval pattern across a segment of the inner surface of the intermediate shell.

5. The joint replacement implant of claim 4, wherein the mobility ridge extends distally from an inner surface of the intermediate shell and forms the oval pattern when projected on a two-dimensional surface.

6. The joint replacement implant of claim 2, wherein the mobility ridge is formed by edges of a void in the partial sphere.

7. The joint replacement implant of claim 5, wherein the void is an oval shape when projected into a two-dimensional plane.

8. The joint replacement implant of claim 1, wherein the intermediate shell and the outer shell interoperate to enable the following rotational ranges of the monoblock neck while preventing dislocation of the ball:
abduction rotation up to 100 degrees;
adduction rotation up to 45 degrees;
internal rotation up to 50 degrees;
external rotation up to 80 degrees;
extension up to 25 degrees; and
flexion up to 150 degrees.

9. The joint replacement implant of claim 8, wherein interaction between the shell mobility structure and the plurality of mobility ridges on the intermediate shell operate to move the intermediate shell to cover the ball to prevent dislocation of the ball.

10. The joint replacement implant of claim 1, wherein an inner surface of the outer shell forms a partial sphere.

11. The joint replacement implant of claim 10, wherein a cross-section of the outer shell forms a semi-circle extending approximately 190 degrees to 220 degrees.

12. The joint replacement implant of claim 1, further comprising a liner disposed between the intermediate shell and the outer shell.

13. The joint replacement implant of claim 12, wherein the liner is a spherical frustum with an inner diameter corresponding to an outer diameter of the intermediate shell and an outer diameter corresponding to an inner diameter of the outer shell.

14. The joint replacement implant of claim 13, wherein the liner comprises a plurality of liner sections that are configured to connect to the intermediate shell, the outer shell, or another of the plurality of liner section.

15. The joint replacement implant of claim 1, wherein the fixation features of the outer shell include a conical portion at a proximal apex.

16. The joint replacement implant of claim 1, further comprising a modular neck couplable to the monoblock neck.

17. The joint replacement implant of claim 16, wherein the modular neck includes an adjustable stem interface to enable femoral anteversion adjustment between the ball and a femoral stem.

18. The joint replacement implant of claim 1, further comprising a sensor in the ball.

19. The joint replacement implant of claim 18, wherein the sensor is an accelerometer or gyroscope.

20. The joint replacement implant of claim 18, wherein the sensor is configured to transmit data corresponding to a range of motion.
